(19) United States
(12) Patent Application Publication
(10) Pub. No.: US 2026/0050327 A1
(43) Pub. Date: Feb. 19, 2026

(54) COMMUNICATION DEVICE AND COMMUNICATION METHOD

(71) Applicant: HTC Corporation, Taoyuan City (TW)

(72) Inventors: Chun-Yih WU, Taoyuan City (TW); Ta-Chun PU, Taoyuan City (TW); Yen-Liang KUO, Taoyuan City (TW)

(73) Assignee: HTC Corporation, Taoyuan City (TW)

(21) Appl. No.: 18/903,698

(22) Filed: Oct. 1, 2024

(30) Foreign Application Priority Data
Aug. 15, 2024 (TW) 113130618

(51) Int. Cl. G06F 3/01; A61B 5/369; G06F 3/16

(52) U.S. Cl. CPC G06F 3/015; A61B 5/369; G06F 3/016; G06F 3/167

(57) ABSTRACT

A communication device includes a pressure sensor, an electrode element, an HBC (Human Body Communication) module, and a processor. The pressure sensor detects a pressure signal from a user. The electrode element is configured to touch the user. The HBC module is coupled to the electrode element. The processor is coupled to the pressure sensor and the HBC module. The processor compares the strength of the pressure signal with a first threshold and a second threshold. If the strength of the pressure signal is greater than the first threshold, the processor will control the HBC module to perform a normal HBC process. If the strength of the pressure signal is decreased to the second threshold, the processor will generate a notification signal.

### CROSS REFERENCE TO RELATED APPLICATIONS

This application claims priority of Taiwan Patent Application No. 113130618 filed on Aug. 15, 2024, the entirety of which is incorporated by reference herein.

### BACKGROUND OF THE INVENTION

### Field of the Invention

The invention relates to a communication device, and more particularly, to a communication device and a communication method.

### Description of the Related Art

In the field of VR (Virtual Reality) and AR (Augmented Reality), one device may be connected to another related device in a wired or wireless manner. However, a general connection tends to have the problem of poor communication quality. Accordingly, there is a need to propose a novel solution for solving this problem of the prior art.

### BRIEF SUMMARY OF THE INVENTION

In an exemplary embodiment, the invention is directed to a communication device for interacting with a user. The communication device includes a pressure sensor, an electrode element, an HBC (Human Body Communication) module, and a processor. The pressure sensor detects a pressure signal from the user. The electrode element is configured to touch the user. The HBC module is coupled to the electrode element. The processor is coupled to the pressure sensor and the HBC module. The processor compares the strength of the pressure signal with a first threshold and a second threshold. If the strength of the pressure signal is greater than the first threshold, the processor will control the HBC module to perform a normal HBC process. If the strength of the pressure signal is decreased to the second threshold, the processor will generate a notification signal.

In some embodiments, the second threshold is lower than the first threshold.

In some embodiments, the electrode element is integrated with the pressure sensor.

In some embodiments, the communication device further includes a vibration module coupled to the processor. The vibration module generates a reminder vibration signal according to the notification signal.

In some embodiments, if the strength of the pressure signal is between the first threshold and the second threshold, the processor will control the HBC module to perform a low-speed HBC process.

In some embodiments, the communication device further includes a wireless communication module coupled to the processor. If the strength of the pressure signal is continuously lower than the second threshold, the processor will stop the low-speed HBC process and control the wireless communication module to perform a wireless communication process.

In some embodiments, before the normal HBC process or the low-speed HBC process is terminated, the processor controls the vibration module to generate an ending vibration signal.

In some embodiments, the communication device further includes a speaker coupled to the processor. Before the normal HBC process or the low-speed HBC process is terminated, the processor controls the speaker to generate an ending sound signal.

In some embodiments, the communication device further includes an EEG (Electroencephalography) module coupled to the processor. Before the normal HBC process is performed, the EEG module receives a setting signal from the user.

In some embodiments, the setting signal includes data relative to a transmission format, an initial transmission rate, and an amplifier supply voltage.

In some embodiments, before the normal HBC process or the low-speed HBC process is terminated, the processor controls the EEG module to generate an ending EEG signal.

In another exemplary embodiment, the invention is directed to a communication method that includes the steps of: detecting a pressure signal from a user by a pressure sensor; touching the user by an electrode element, wherein an HBC module is coupled to the electrode element; comparing a strength of the pressure signal with a first threshold and a second threshold; if the strength of the pressure signal is greater than the first threshold, controlling the HBC module to perform a normal HBC process; and if the strength of the pressure signal is decreased to the second threshold, generating a notification signal.

In some embodiments, the communication method further includes: integrating the electrode element with the pressure sensor.

In some embodiments, the communication method further includes: generating a reminder vibration signal according to the notification signal by a vibration module.

In some embodiments, the communication method further includes: if the strength of the pressure signal is between the first threshold and the second threshold, controlling the HBC module to perform a low-speed HBC process.

In some embodiments, the communication method further includes: if the strength of the pressure signal is continuously lower than the second threshold, stopping the low-speed HBC process and controlling a wireless communication module to perform a wireless communication process.

In some embodiments, the communication method further includes: before the normal HBC process or the low-speed HBC process is terminated, controlling the vibration module to generate an ending vibration signal.

In some embodiments, the communication method further includes: before the normal HBC process or the low-speed HBC process is terminated, controlling a speaker to generate an ending sound signal.

In some embodiments, the communication method further includes: before the normal HBC process is performed, receiving a setting signal from the user by an EEG module.

In some embodiments, the communication method further includes: before the normal HBC process or the low-speed HBC process is terminated, controlling the EEG module to generate an ending EEG signal.

### BRIEF DESCRIPTION OF DRAWINGS

The invention can be more fully understood by reading the subsequent detailed description and examples with references made to the accompanying drawings, wherein:

FIG. 1 is a diagram of a communication device according to an embodiment of the invention;

FIG. 2 is a diagram of a communication device according to an embodiment of the invention;

FIG. 3 is a diagram of a waveform of a detected pressure signal of a communication device according to an embodiment of the invention;

FIG. 4 is a diagram of a communication device according to an embodiment of the invention;

FIG. 5 is a diagram of a waveform of a detected pressure signal of a communication device according to an embodiment of the invention;

FIG. 6 is a diagram of a communication device according to an embodiment of the invention; and

FIG. 7 is a flowchart of a communication method according to an embodiment of the invention.

### DETAILED DESCRIPTION OF THE INVENTION

In order to illustrate the foregoing and other purposes, features and advantages of the invention, the embodiments and figures of the invention will be described in detail as follows.

Certain terms are used throughout the description and following claims to refer to particular components. As one skilled in the art will appreciate, manufacturers may refer to a component by different names. This document does not intend to distinguish between components that differ in name but not function. In the following description and in the claims, the terms “include” and “comprise” are used in an open-ended fashion, and thus should be interpreted to mean “include, but not limited to.” . The term “substantially” means the value is within an acceptable error range. One skilled in the art can solve the technical problem within a predetermined error range and achieve the proposed technical performance. Also, the term “couple” is intended to mean either an indirect or direct electrical connection. Accordingly, if one device is coupled to another device, that connection may be through a direct electrical connection, or through an indirect electrical connection via other devices and connections.

The following disclosure provides many different embodiments, or examples, for implementing different features of the subject matter provided. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

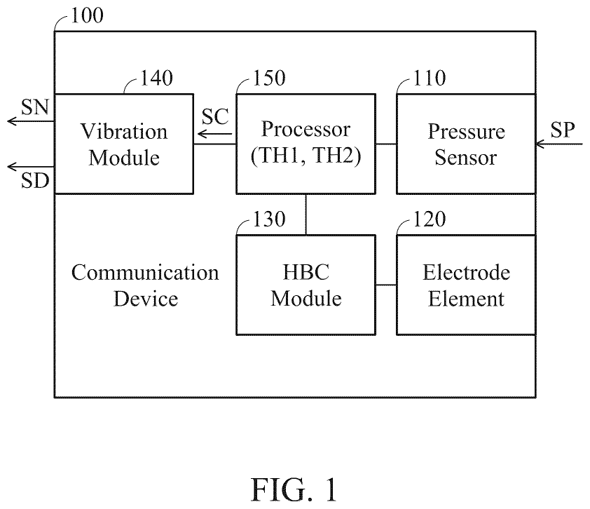

FIG. 1 is a diagram of a communication device 100 according to an embodiment of the invention. For example, the communication device 100 may be applied to relative equipment of VR (Virtual Reality) or AR (Augmented Reality). Alternatively, the communication device 100 may be applied to a mobile device, such as a smart phone, a tablet computer, or a notebook computer. As shown in FIG. 1, the communication device 100 at least includes a pressure sensor 110, an electrode element 120, an HBC (Human Body Communication) module 130, and a processor 150. It should be understood that the communication device 100 may include other components, such as a battery, a touch control panel, a power supply module and/or a housing, although they are not displayed in FIG. 1.

The communication device 100 can interact with a user HB. The pressure sensor 110 can detect a pressure signal SP from the user HB. For example, the electrode element 120 may include one or more metal strips, but it is not limited thereto. The electrode element 120 is configured to touch any portion of the body of the user HB. In some embodiments, the electrode element 120 is integrated with the pressure sensor 110. The HBC module 130 is coupled to the electrode element 120. The processor 150 is coupled to the pressure sensor 110 and the HBC module 130. The processor 150 can compare the strength of the pressure signal SP with a first threshold TH1 and a second threshold TH2. For example, the second threshold TH2 may be lower than the first threshold TH1, and they are previously stored or adjustable. If the strength of the pressure signal SP is greater than the first threshold TH1, the processor 150 will control the HBC module 130 to perform a normal HBC process. In some embodiments, the HBC module 130 can quickly collect a variety of physiological information from the user HB by using the electrode element 120. If the strength of the pressure signal SP is decreased to the second threshold TH2, the processor 150 will generate a notification signal SC. For example, the notification signal SC may be transmitted to another element based on a wired mechanism or a wireless mechanism, but it is not limited thereto.

In some embodiments, the communication device 100 further includes a vibration module 140 coupled to the processor 150. The vibration module 140 can generate a reminder vibration signal SN according to the notification signal SC. For example, the reminder vibration signal SN may be configured to remind the user HB of further increasing the strength of the pressure signal SP. It should be understood that the vibration module 140 is merely an optional element, which is removable from the communication device 100 in other embodiments. In alternative embodiments, the vibration module 140 is disposed in an external device (not shown), and the external device is independent of the communication device 100.

With the design of the invention, the proposed communication device 100 can communicate with the user HB or its relative device (not shown) by using an HBC mechanism. When the strength of the pressure signal SP is insufficient, the proposed communication device 100 can remind the user HB of appropriately adjusting it, so as to maintain the overall good communication quality.

The following embodiments will introduce different configurations and detail structural features of the communication device 100. It should be understood that these figures and descriptions are merely exemplary, rather than limitations of the invention.

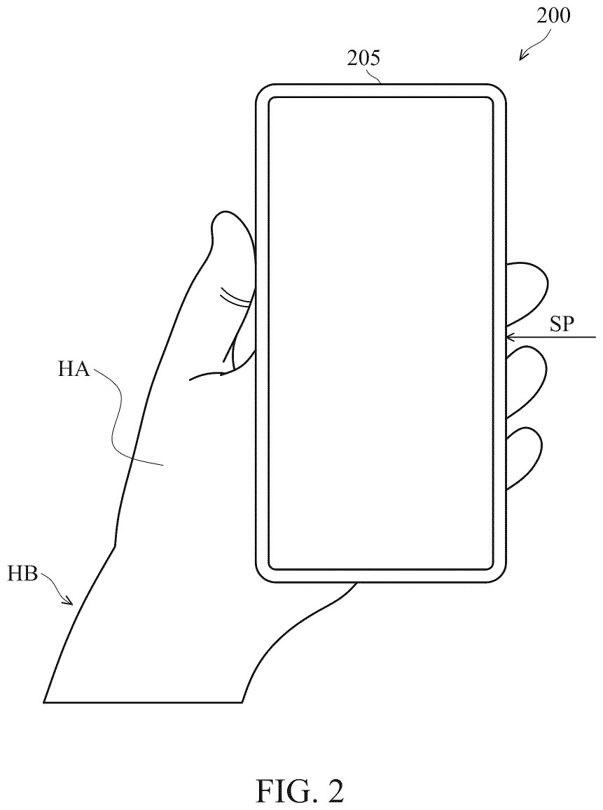

FIG. 2 is a diagram of a communication device 200 according to an embodiment of the invention. FIG. 2 is similar to FIG. 1. In the embodiment of FIG. 2, the communication device 200 is a smart phone and further includes a nonconductive housing 205. For example, when the communication device 200 is tightly held by a hand portion HA of the user HB, the strength of the corresponding pressure signal SP may be increased, such that the communication quality of the relative HBC mechanism may be improved. Conversely, when the communication device 200 is gently held by the hand portion HA of the user HB, the strength of the corresponding pressure signal SP may be decreased. For example, an electrode element of the communication device 200 may be disposed on an outer side of the nonconductive housing 205, and a pressure sensor of the communication device 200 may be disposed on an inner side of the nonconductive housing 205 (not shown). Alternatively, both the pressure sensor and the electrode element of the communication device 200 may be disposed on the same side of the nonconductive housing 205. Other features of the communication device 200 of FIG. 2 are similar to those of the communication device 100 of FIG. 1. As a result, the two embodiments can achieve similar levels of performance.

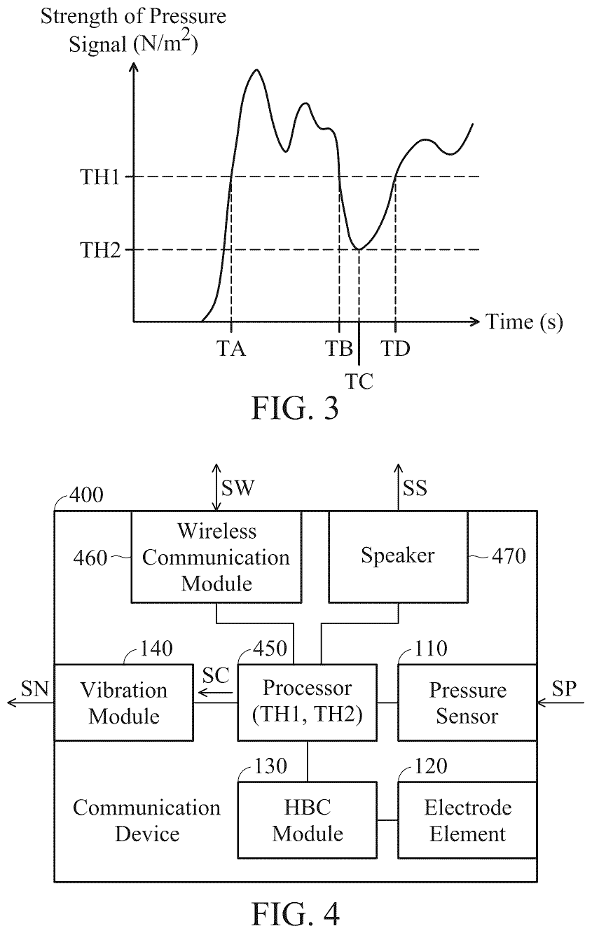

FIG. 3 is a diagram of the waveform of the detected pressure signal SP of the communication device 100 according to an embodiment of the invention. The horizontal axis represents time(s), and the vertical axis represents the strength of the pressure signal SP (N/m²). Please refer to FIG. 1 and FIG. 3 together. At a first time point TA, the strength of the pressure signal SP is increased to the first threshold TH1, and thus the processor 150 can control the HBC module 130 to perform a normal HBC process. At a second time point TB, the strength of the pressure signal SP is decreased back to the first threshold TH1. Next, at a third time point TC, the strength of the pressure signal SP is further decreased to the second threshold TH2. In some embodiments, if the strength of the pressure signal SP is between the first threshold TH1 and the second threshold TH2 (i.e., during the period from the second time point TB to the third time point TC), the processor 150 can control the HBC module 130 to perform a low-speed HBC process, so as to replace the aforementioned normal HBC process. For example, the transmission rate of the low-speed HBC process may be lower than that of the normal HBC process. It should be noted that at the third time point TC, the processor 150 can also control the vibration module 140 to output the reminder vibration signal SN for reminding the user HB. In response to the reminder vibration signal SN, the user HB can increase the strength of the pressure signal SP again. Finally, at a fourth time point TD, the strength of the pressure signal SP is increased back to the first threshold TH1, and the processor 150 can control the HBC module 130 to perform the normal HBC process again, so as to replace the aforementioned low-speed HBC process. In alternative embodiments, before the normal HBC process or the low-speed HBC process is terminated, the processor 150 can control the vibration module 140 to generate an ending vibration signal SD. It should be noted that the ending vibration signal SD is different from the reminder vibration signal SN. For example, the reminder vibration signal SN may be a relatively short vibration, and the ending vibration signal SD may be a relatively long vibration, but they are not limited thereto. In response to the ending vibration signal SD, the user HB can understand that the normal HBC process or the low-speed HBC process is going to be terminated, and can prepare to release the hand portion HA.

FIG. 4 is a diagram of a communication device 400 according to an embodiment of the invention. FIG. 4 is similar to FIG. 1. In the embodiment of FIG. 4, the communication device 400 further includes a wireless communication module 460 and a speaker 470. A processor 450 of the communication device 400 is also coupled to the wireless communication module 460 and the speaker 470. For example, the wireless communication module 460 may be a Wi-Fi module or an LTE (Long Term Evolution) module, but it is not limited thereto. Before a normal HBC process or a low-speed HBC process of the communication device 400 is terminated, the processor 450 can control the speaker 470 to generate an ending sound signal SS. In response to the ending sound signal SS, the user HB can understand that the normal HBC process or the low-speed HBC process is going to be terminated, and can prepare to release the hand portion HA.

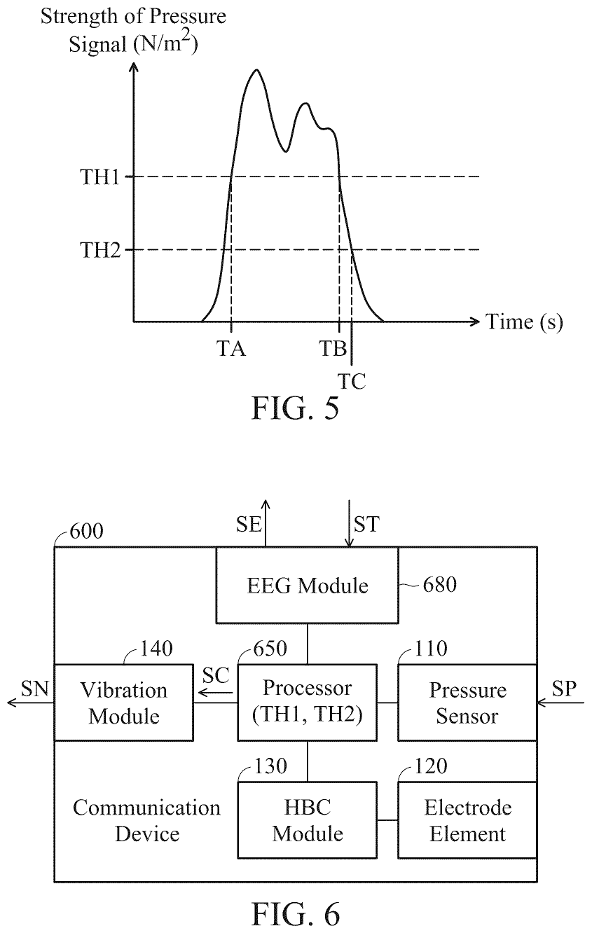

FIG. 5 is a diagram of the waveform of the detected pressure signal SP of the communication device 100 according to an embodiment of the invention. The horizontal axis represents time(s), and the vertical axis represents the strength of the pressure signal SP (N/m²). Please refer to FIG. 4 and FIG. 5 together. After the third time point TC, if the strength of the pressure signal SP is continuously lower than the second threshold TH2, the processor 450 will stop the aforementioned low-speed HBC process and control the wireless communication module 460 to perform a wireless communication process. The wireless communication process may mean the reception or transmission of a wireless signal SW. In some embodiments, the user HB wears a wearable device with a function of wireless communication (not shown), such as a pair of VR glasses. Next, the communication device 400 can communicate with the wearable device of the user HB by using a wireless communication mechanism, so as to replace the aforementioned HBC mechanism. According to practical measurements, the incorporation of the wireless communication mechanism can help to enhance the diversity design of the communication device 400. Other features of the communication device 400 of FIG. 4 are similar to those of the communication device 100 of FIG. 1. As a result, the two embodiments can achieve similar levels of performance.

FIG. 6 is a diagram of a communication device 600 according to an embodiment of the invention. FIG. 6 is similar to FIG. 1. In the embodiment of FIG. 6, the communication device 600 further includes an EEG (Electroencephalography) module 680, which is coupled to a processor 650 of the communication device 600. The user HB can wear another wearable device with an EEG function (not shown), such as an HMD (Head Mounted Display). Before a normal HBC process of the wearable device 600 is performed, the EEG module 680 can receive a setting signal ST from the user HB and the wearable device thereof. Specifically, the setting signal ST may include the data relative to a transmission format, an initial transmission rate, and an amplifier supply voltage. Based on the setting signal ST, the normal HBC process and a low-speed HBC process of the communication device 600 can be optimized. In addition, before the normal HBC process or the low-speed HBC process of the communication device 600 is terminated, the processor 650 can control the EEG module 680 to generate an ending EEG signal SE. In response to the ending EEG signal SE, the user HB can understand that the normal HBC process or the low-speed HBC process is going to be terminated, and can prepare to release the hand portion HA. Other features of the communication device 600 of FIG. 6 are similar to those of the communication device 100 of FIG. 1. As a result, the two embodiments can achieve similar levels of performance.

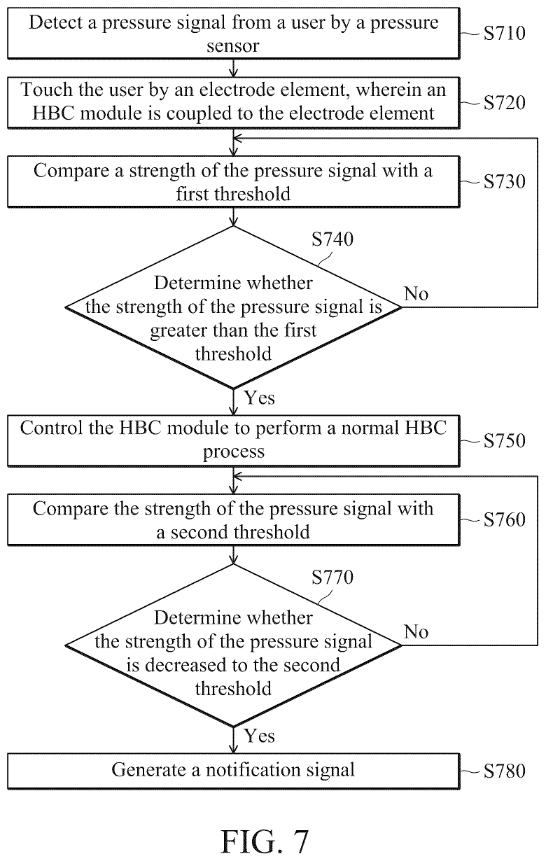

FIG. 7 is a flowchart of a communication method according to an embodiment of the invention. To begin, in step S710, a pressure signal from a user is detected by a pressure sensor. In step S720, the user is touched by an electrode element, and an HBC module is coupled to the electrode element. In step S730, the strength of the pressure signal is compared with a first threshold. In step S740, it is determined whether the strength of the pressure signal is greater than the first threshold. If not, the procedure will go back to step S730. If so, in step S750, the HBC module is controlled to perform a normal HBC process. During the normal HBC process, the strength of the pressure signal may fluctuate upwardly and downwardly. In step S760, the strength of the pressure signal is compared with a second threshold. In step S770, it is determined whether the strength of the pressure signal is decreased to the second threshold. If not, the procedure will go back to step S760. If so, in step S780, a notification signal will be generated. It should be understood that these steps are not required to be performed in order, and every feature of the embodiments of FIGS. 1-6 may be applied to the communication method of FIG. 7.

The invention proposes a novel communication device and a novel communication method. In comparison to the conventional design, the invention has at least the advantages of improving the overall communication quality. Therefore, the invention is suitable for application in a variety of devices.

Note that the above element parameters are not limitations of the invention. A designer can fine-tune these setting values according to different requirements. It should be understood that the communication device and the communication method of the invention are not limited to the configurations of FIGS. 1-7. The invention may include any one or more features of any one or more embodiments of FIGS. 1-7. In other words, not all of the features displayed in the figures should be implemented in the communication device and the communication method of the invention.

The method of the invention, or certain aspects or portions thereof, may take the form of program code (i.e., executable instructions) embodied in tangible media, such as floppy diskettes, CD-ROMS, hard drives, or any other machine-readable storage medium, wherein, when the program code is loaded into and executed by a machine such as a computer, the machine thereby becomes an apparatus for practicing the methods. The methods may also be embodied in the form of program code transmitted over some transmission medium, such as electrical wiring or cabling, through fiber optics, or via any other form of transmission, wherein, when the program code is received and loaded into and executed by a machine such as a computer, the machine becomes an apparatus for practicing the disclosed methods. When implemented on a general-purpose processor, the program code combines with the processor to provide a unique apparatus that operates analogously to application-specific logic circuits.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be apparent to those skilled in the art that various modifications and variations can be made in the invention. It is intended that the standard and examples be considered as exemplary only, with a true scope of the disclosed embodiments being indicated by the following claims and their equivalents.

1. A communication device for interacting with a user, comprising:
a pressure sensor, detecting a pressure signal from the user;
an electrode element, configured to touch the user;
an HBC (Human Body Communication) module, coupled to the electrode element; and
a processor, coupled to the pressure sensor and the HBC module, wherein the processor compares a strength of the pressure signal with a first threshold and a second threshold;
wherein if the strength of the pressure signal is greater than the first threshold, the processor controls the HBC module to perform a normal HBC process;
wherein if the strength of the pressure signal is decreased to the second threshold, the processor generates a notification signal;
wherein the electrode element is integrated with the pressure sensor.

2. The communication device as claimed in claim 1, wherein the second threshold is lower than the first threshold.

3. (canceled)

4. The communication device as claimed in claim 1, further comprising:
a vibration module, coupled to the processor, wherein the vibration module generates a reminder vibration signal according to the notification signal.

5. The communication device as claimed in claim 4, wherein if the strength of the pressure signal is between the first threshold and the second threshold, the processor controls the HBC module to perform a low-speed HBC process.

6. The communication device as claimed in claim 5, further comprising:
a wireless communication module, coupled to the processor, wherein if the strength of the pressure signal is continuously lower than the second threshold, the processor stops the low-speed HBC process and controls the wireless communication module to perform a wireless communication process.

7. The communication device as claimed in claim 5, wherein before the normal HBC process or the low-speed HBC process is terminated, the processor controls the vibration module to generate an ending vibration signal.

8. The communication device as claimed in claim 5, further comprising:
a speaker, coupled to the processor, wherein before the normal HBC process or the low-speed HBC process is terminated, the processor controls the speaker to generate an ending sound signal.

9. The communication device as claimed in claim 5, further comprising:
an EEG (Electroencephalography) module, coupled to the processor, wherein before the normal HBC process is performed, the EEG module receives a setting signal from the user.

10. The communication device as claimed in claim 9, wherein the setting signal comprises data relative to a transmission format, an initial transmission rate, and an amplifier supply voltage.

11. The communication device as claimed in claim 9, wherein before the normal HBC process or the low-speed HBC process is terminated, the processor controls the EEG module to generate an ending EEG signal.

12. A communication method, comprising the steps of:
detecting a pressure signal from a user by a pressure sensor;
touching the user by an electrode element, wherein an HBC module is coupled to the electrode element;
comparing a strength of the pressure signal with a first threshold and a second threshold;
integrating the electrode element with the pressure sensor;
if the strength of the pressure signal is greater than the first threshold, controlling the HBC module to perform a normal HBC process; and
if the strength of the pressure signal is decreased to the second threshold, generating a notification signal.

13. The communication method as claimed in claim 12, wherein the second threshold is lower than the first threshold.

14. (canceled)

15. The communication method as claimed in claim 12, further comprising:
generating a reminder vibration signal according to the notification signal by a vibration module.

16. The communication method as claimed in claim 15, further comprising:
if the strength of the pressure signal is between the first threshold and the second threshold, controlling the HBC module to perform a low-speed HBC process.

17. The communication method as claimed in claim 16, further comprising:
if the strength of the pressure signal is continuously lower than the second threshold, stopping the low-speed HBC process and controlling a wireless communication module to perform a wireless communication process.

18. The communication method as claimed in claim 16, further comprising:
before the normal HBC process or the low-speed HBC process is terminated, controlling the vibration module to generate an ending vibration signal.

19. The communication method as claimed in claim 16, further comprising:
before the normal HBC process or the low-speed HBC process is terminated, controlling a speaker to generate an ending sound signal.

20. The communication method as claimed in claim 16, further comprising:
before the normal HBC process is performed, receiving a setting signal from the user by an EEG module.

21. The communication method as claimed in claim 20, wherein the setting signal comprises data relative to a transmission format, an initial transmission rate, and an amplifier supply voltage.

22. The communication method as claimed in claim 20, further comprising:
before the normal HBC process or the low-speed HBC process is terminated, controlling the EEG module to generate an ending EEG signal.
